(19) United States
(12) Patent Application Publication
(10) Pub. No.: US 2023/0296602 A1
(43) Pub. Date: Sep. 21, 2023

(54) ASSAY METHOD, KIT, AND REAGENTS FOR QUANTITATIVE DETERMINATION OF ANTIBODIES AGAINST SELECTED VIRUSES

(71) Applicant: Gentian AS, Moss (NO)

(72) Inventors: David ARNLUND, Lerum (SE); Camilla FANT, Kullavik (SE); Aleksandra Mandic HAVELKA, Saltsjö Boo (SE); Clara Mathilde HIDDEN, Vestby (NO); Torsten KNÜTTEL, Ski (NO); Evelina LE COMTE, Partille (SE); Tom NILSEN, Moss (NO); Erling SUNDREHAGEN, Moss (NO)

(21) Appl. No.: 17/926,255

(22) PCT Filed: May 20, 2021
(86) PCT No.: PCT/EP2021/063429

§ 371 Date: Nov. 18, 2022

(30) Foreign Application Priority Data
May 20, 2020 (SE) 2050593-9

(51) Int. Cl. G01N 33/569; G01N 33/58

(52) U.S. Cl. CPC G01N 33/56983; G01N 33/587; G01N 2333/165; G01N 2469/20; G01N 2470/06

(57) ABSTRACT

Antibodies against a virus can be detected in a sample using at least one reagent comprising at least one capture molecule immobilized to a particle, which reagent in the presence of such antibodies forms an anti-virus antibody-capture molecule complex, wherein the presence of said complex is qualitatively, quantitatively or semi-quantitatively determined by measuring a signal generated by said complex, said particle is a nanoparticle and said capture molecule is immobilized to said nanoparticle simultaneously with a co-molecule which is smaller than said capture molecule. When the capture molecule is a virus epitope, the co-molecule preferably has a molecular weight in the range of 50-1500 Da, wherein said co-molecules when immobilized on said nanoparticle separate the capture molecules so that an average distance between two adjacent capture molecules is greater than a distance between the antigen binding sites of the anti-virus antibody to be detected.

### TECHNICAL FIELD

The present disclosure relates to the field of diagnostic assays, and in particular to methods, kits and reagents for determining the presence or concentration of antibodies in a sample of a human or mammalian body fluid or a fluid sample extracted from mammalian biological material, for example for the detection of antibodies against a virus belonging to the family of coronaviruses.

### BACKGROUND

Epidemics caused by viruses have been around long before the existence of viruses was even known to man, and most likely throughout the human evolutionary history. Measles and smallpox are among the oldest viruses known to infect humans, and influenza pandemics have been recorded since the middle ages. The influenza pandemic around 1918-19 known as the Spanish flu, and the poliomyelitis epidemic in the first half of the 20th century created a general awareness of the dangers of viruses, and during the rest of the 20th century, advances in science have resulted in a better understanding of viral diseases and also led to a scientific, reliable and efficient development of vaccines. Nevertheless, humanity is still severely tormented by viral outbreaks, as demonstrated by the outbreak of the SARS-CoV-2 virus, first identified in Wuhan, China, in December 2019.

Methods for rapid and reliable detection of viral infections are important in the control of infectious diseases, such as respiratory and gastrointestinal infections. The outbreak of the global pandemic caused by SARS-CoV-2 has increased the need for methods allowing cost-efficient yet reliable mass testing. The current approaches can be divided into two main groups; serology tests, which detect the presence of antibodies produced in response to viral infection; and tests that detect the presence of the virus itself.

Testing for the presence of the virus itself is almost exclusively based on the detection of virus nucleic acid using a polymerase chain reaction (PCR) to selectively amplify specific parts of the virus genome until they reach a detectable level. The advantage of PCR based tests is their high sensitivity, and some tests are reported to be able to detect even 0.05 viral copies per cell. The sensitivity and reliability of PCR based tests is however strictly dependent on the choice of primers, as these decide which part of the virus genome will be amplified.

Today, PCR analysis has become mainstream work in clinical laboratories, and the time for completing an analysis typically involving 40 cycles has been reduced to about one hour. While this represents a significant improvement, PCR analysis is still rather time-consuming and not ideally suited for mass testing. Additionally, PCR analysis can detect the presence of a virus at the time of sampling but does not give any information about past infections and the possible development of immunity.

Serology tests, i.e. the indirect diagnosis based on the detection of antibodies, typically from different immunoglobulin classes, most commonly from the immunoglobulin classes IgG, IgM and IgA, are important as they give valuable information also after an infection, and can be used to monitor the spread of an infection in a population and predicting the future development of a pandemic based on the degree of immunity. Serology tests can also be useful in vaccine research and development, and for monitoring the results of a vaccination regime.

An early disclosure of a serology test performed by turbidimetry is found in the article “Turbidimetric Method for the Assay of Antiviral Antibodies” (Dandliker et al., Journal of Virology, March 1969, p. 283-289). In this method, turbidity changes effected by a virus-antibody reaction were followed by optical density measurements at 436 nm using a spectrophotometer.

There remains however a need for automated testing for detecting the presence of antibodies against disease-causing viruses, and in particular against highly contagious viruses, such as but not limited to viruses belonging to the family of corona viruses, for example the SARS-CoV-2 virus.

Measurement of antibodies created in an organism as a response to infection has traditionally been assayed by so-called heterogeneous immunoassays. Such heterogeneous measurement is typically based on directly or indirectly coating the virus, modified virus, part of the virus or a conjugate of a virus to a solid phase, incubating the solid phase with a sample known or suspected to comprise antibodies, under conditions allowing for binding of antibodies to said viruses and or fragments or conjugates (hereafter called virus and analogues), and directly or indirectly detecting the anti-virus antibodies bound to said solid phase.

Another assay format is the so-called double antibody bridge assay, where the assay detects primary antibodies from the patient samples bound to an antigen structure on a solid phase used in the assay, using secondary antibodies with signal-generating moieties such as enzymes, fluorophores and other signal-generating moieties. In such heterogeneous immunoassays, several washing steps are needed. Although these methods have many qualities, they are relatively slow and demand sophisticated instrumentation both for washing and for the detection, e.g. for detection of an enzymatic reaction, for measurement of fluorescence or chemiluminescence. There is therefore a need for simpler, faster, and less expensive methods for the detection and specific quantitation of antibodies against viruses in biological samples.

### SUMMARY

According to a first aspect, the present description makes available a method for detection of an antibody against a virus or viral antigens in a sample suspected of comprising antibodies against said virus, comprising:
contacting the sample with a nanoparticle having capture molecules and co-molecules immobilized thereon, wherein a capture molecule comprises a peptide that is specifically recognized by the antibody and wherein a co-molecule is not specifically recognized by the antibody and has a molecular weight smaller than the molecular weight of the capture molecule,
forming a complex of the antibody with the capture molecule immobilized on the nanoparticle in the presence of the antibodies in the sample, and
determining a signal indicative for the presence of the complex.

According to an embodiment said capture molecule is an epitope isolated from a spike, envelope, membrane or nucleocapsid protein of said virus.

The method is applicable to any virus, but according to an embodiment currently preferred by the inventors, said virus is a SARS-CoV-2 virus including variants thereof.

According to an embodiment of said first aspect, the co-molecules when immobilized on said nanoparticle separate the capture molecules so that an average distance between two adjacent capture molecules is greater than a distance between antigen binding sites of the anti-virus antibody to be detected.

According to an embodiment, the co-molecule has a primary amine moiety.

According to an embodiment, the co-molecule is chosen from the group comprising hydroxylamine, tris(hydroxymethyl)aminomethane, amino acids, low molecular weight peptides and ethanolamine.

According to a preferred embodiment, the co-molecule is ethanolamine.

According to an embodiment, wherein the co-molecule has a molecular weight in the range of 50-1500 Da. For future virus variants, and different capture molecules, the molecular weight of the co-molecule can be adjusted accordingly.

According to an embodiment, the co-molecule is a peptide having a molecular weight in the range of 300-1500 Da. For future virus variants, and different capture molecules, the molecular weight of the co-molecule can be adjusted accordingly.

According to an embodiment, the signal is measured by turbidimetry or nephelometry.

According to an embodiment, the sample is a sample chosen from mammalian body fluids such as mucus, including nasal or laryngeal mucus, sputum, saliva, tears, feces, including fecal extracts, urine, whole blood or a blood derived sample like blood plasma or blood serum.

According to a preferred embodiment, the sample is a sample chosen from mammalian blood plasma and serum.

According to an embodiment of the first aspect and freely combinable with any embodiments mentioned herein, the method further comprises a calibration step using calibration samples having an antibody content ranging from 0 to 100 μg/ml of anti-SARS-CoV-2 IgG antibody in plasma. When determining the concentration of IgM, the method further comprises a calibration step using calibration samples having an antibody content ranging from 0 to 200 μg/ml of anti-SARS-CoV-2 IgM antibody in plasma. For IgA, the corresponding calibration range may be 0-100 μg/ml in plasma.

In the context of this disclosure, any anti-virus antibodies can be detected, qualitatively, quantitatively or semi-quantitatively, depending on the choice of the capture molecule. In the light of the pandemic ravaging the world at the priority date of the present disclosure, the present inventors have primarily focused on antibodies specific to a virus belonging to the corona virus family, for example but not limited to the currently known human coronavirus types 229E (alpha coronavirus), NL63 (alpha coronavirus), 0043 (beta coronavirus), HKU1 (beta coronavirus), MERS-CoV (a beta coronavirus that causes the Middle East Respiratory Syndrome, MERS), SARS-CoV (a beta coronavirus that causes severe acute respiratory syndrome, SARS), and SARS-CoV-2 and variants thereof, such as the novel coronavirus that causes coronavirus disease 2019, COVID-19.

Preferably said antibodies are antibodies directed against an epitope isolated from the virus spike, envelope, membrane or nucleocapsid proteins. It is however contemplated that other viral proteins or peptides are identified, and in the following description, all potential antigens are referred to as “viral antigens”, abbreviated “VA” in the figures.

According to a preferred embodiment, said antibodies are antibodies against a viral antigen specific for viruses belonging to the family of corona viruses, and preferably a viral antigen which alone or in combination with other viral antigens is specific for an individual corona virus, such as SARS-CoV-2. Specificity for an individual virus here means that the assay exhibits no or negligible cross reactivity with other related viruses. Preferably said antibodies are antibodies directed against a conserved epitope specific for SARS-CoV-2, for example a conserved epitope isolated from the SARS-CoV-2 spike protein, such as “51” but other viral antigens are also contemplated.

The turbidimetric or nephelometric methods according to any embodiment above may additionally comprise a step of adding an opacity enhancer, for example by including an opacity enhancer in one or more of the reagents used in the method.

The concentration of antibodies is determined as antibody titer, and can be reported as antibody units (AU)/ml, or as μg/ml or mg/L. Generally, antibody titer is defined as the greatest dilution (lowest concentration) of the blood sample at which an antibody assay, such as ELISA, still produces a detectable positive result. The higher the antibody concentration in the blood, the greater the dilution that will produce a detectable signal. The actual titer value for an antibody will however vary based on the antibody being tested, the method used, and the laboratory performing the test.

Another aspect of the disclosure is a kit for performing a method according to the first aspect and any embodiments thereof. Thus, the present disclosure makes available a kit for detection of antibodies against a virus in a sample, comprising at least one specific capture molecule for an anti-virus antibody and a co-molecule immobilized to a nanoparticle, wherein
said capture molecule comprises an epitope isolated from a spike, envelope, membrane or nucleocapsid protein of said virus,
said co-molecule is not specifically recognized by the antibody and has a molecular weight smaller than that of the capture molecule,
said co-molecules when immobilized on said nanoparticle separate the capture molecules so that an average distance between two adjacent capture molecules is greater than a distance between the antigen binding sites of the anti-virus antibody to be detected.

According to an embodiment of said second aspect, the capture molecule comprises an epitope isolated from a spike protein of a SARS-CoV-2 virus or variants thereof.

According to another embodiment of said second aspect, freely combinable with the above, the co-molecule has a primary amine moiety.

Preferably the co-molecule is chosen from the group comprising hydroxylamine, tris(hydroxymethyl)aminomethane, amino acids, low molecular weight peptides and ethanolamine. According to a preferred embodiment, the co-molecule is ethanolamine.

The co-molecule then preferably has a molecular weight in the range of 50-1500 Da. For future virus variants, and when using different capture molecules, the molecular weight of the co-molecule can be adjusted accordingly.

According to an alternative embodiment of the second aspect, the co-molecule is a low-molecular weight peptide, preferably a peptide having a molecular weight in the range of 300-1500 Da. For future virus variants, and when using different capture molecules, the molecular weight of the co-molecule is adjusted accordingly.

According to another embodiment of the second aspect and freely combinable with any embodiments mentioned herein, the kit further comprises a set of calibrators having an antibody content ranging from 0 to 200 μg/ml, e.g. 0 to 20 μg/ml of anti-When determining the concentration of IgM, the kit further comprises a set of calibrators having antibody contents ranging from 0 to 100 μg/ml of anti-SARS-CoV-2 antibody (IgG) in plasma. When the method is adapted to quantify IgM, the same or a different clinical range will be used, for example 0-200 μg/ml IgM, and for IgA, 0-100 μg/ml is contemplated.

According to another embodiment of the second aspect and freely combinable with any embodiments mentioned herein, said nanoparticle has a diameter in the range of 10-300 nm.

A kit according to the second aspect or any embodiment thereof may optionally include auxiliary reagents for performing the measurement.

The invention also makes available a nanoparticle for use in a method according to the first aspect and any embodiments thereof, and/or as part of a kit according to the second aspect and any embodiments thereof, comprising
a capture molecule chosen from epitopes isolated from a spike, envelope, membrane or nucleocapsid protein of a virus, and a co-molecule immobilized to said nanoparticle, wherein
said co-molecule is not specifically recognized by the antibody and has a molecular weight which is smaller than that of said capture molecule, and wherein
said co-molecules when immobilized on said particles separate the capture molecules so that an average distance between two adjacent capture molecules is greater than a distance between the antigen binding sites of the anti-virus antibody to be detected.

In a nanoparticle according to this third aspect of the disclosure, the capture molecule is an epitope isolated from a spike protein of a SARS-CoV-2 virus or variants thereof.

According to an embodiment of the third aspect, the co-molecule molecule has a primary amine moiety. Preferably said co-molecule is chosen from the group comprising hydroxylamine, tris(hydroxymethyl)aminomethane, amino acids, low molecular weight peptides and ethanolamine, and most preferably the co-molecule is ethanolamine.

According to an embodiment, the co-molecule preferably has a molecular weight in the range of 50-1500 Da. For future virus variants, and when using different capture molecules, the molecular weight of the co-molecule is adjusted accordingly.

According to an alternative embodiment, the co-molecule is a low-molecular weight peptide, for example a peptide having a molecular weight in the range of 300-1500 Da. For future virus variants, and when using different capture molecules, the molecular weight of the co-molecule is adjusted accordingly.

### SHORT DESCRIPTION OF THE FIGURES

Different aspects and embodiments of the invention or inventions disclosed herein will be are presented in closer detail in the following description, examples and claims, and in the attached drawings, in which:

FIG. 1 schematically shows crosslinking between anti-viral antibodies in a sample, e.g. a serum sample, and biotin labelled antigenic virus material, e.g. a virus antigen (VA) immobilized to suitable nanoparticles (P). The anti-viral antibodies (anti-VA) bind to virus antigens (VA) immobilized on the particles, and causes aggregation, the degree of which can be measured by measuring transmission or scattering of light of a suitable wavelength, and correlated to the concentration of antibodies in the sample.

FIG. 2 schematically shows an alternative approach, where nanoparticles (P) are coated with virus antigen (VA) bound to secondary antibodies (in bold).

FIG. 3 schematically shows the crosslinking by antibodies from sample material specific for antigenic virus material (VA) on the assay particle reagents (P). The inventors found that the spacing of the capture molecule (virus antigen) on the particles is important, in particular when the capture molecule/virus antigen is a low molecular weight molecule, for example a small peptide, and that steric hindrance as well as too dense packing will negatively influence the assay results.

FIG. 4A schematically shows how dense packing of viral antigens (VA) on particles (P) may lead to the same antibody (anti-VA) binding to two viral antigens on the same particle, thus resulting in less aggregation of particles.

FIG. 4B schematically shows how the use of a conventional co-molecule, here exemplified by ovalbumin (OVA), may create steric hindrance, blocking the antibodies from contacting the antigen. This is the case particularly when antigens of a small molecular weight are used, and where the co-molecule has a molecular weight similar to, or larger than the antigen.

FIG. 5 schematically shows how a co-molecule (CoM, shown here as small circles), which has a smaller molecular weight than the antigen, according to the present disclosure, can be used to regulate the distance/spacing of the antigens so that optimal binding is achieved. The size (molecular weight) and concentration of co-molecules is chosen so that the average distance between individual virus antigens (VA) on the particle (P) is larger than the distance between the binding regions or “arms” of the relevant antibody (anti-VA), without creating steric hindrance for the antibodies to reach and bind to the antigens.

FIG. 6 is a graph showing the results for three different baches measured as relative response in response units (RLU) on the Y-axis, and IgG (μg/ml) on the X-axis. The responses are adjusted to show 0 RU for the lowest concentration for comparison. Results are shown for concentrations ranging from 0-53 μg/ml. The batches were produced using anti-IgG:ovalbumin at a ratio 1:1 (weight to weight, lower curve), anti-IgG:ethanolamine 1:1 (molar ratio, middle curve) and anti-IgG:ethanolamine 1:19 (molar ratio, upper curve). It is evident that exchanging the co-protein ovalbumin into a low Mw molecule containing a primary amine moiety, greatly improves the turbidimetric signal vs concentration and extends the measurement range for the analyte (i.e., later hook).

FIG. 7 is a graph where the measured Covid-19 antibody concentration (mg/L) is plotted against the expected antibody concentration for two measurement series, one performed on untreated samples (marked with circles), and one performed on samples diluted 1:10 (marked with squares). The results indicate that the inventive assay can manage a hook sample up to 100 mg/L.

### DESCRIPTION

Before the present invention is described, it is to be understood that the terminology employed herein is used for the purpose of describing particular embodiments only and is not intended to be limiting, since the scope of the present invention will be limited only by the appended claims and equivalents thereof.

It must be noted that, as used in this specification and the appended claims, the singular forms “a,” “an,” and “the” include plural referents unless the context clearly dictates otherwise.

The term “sample” refers to any sample of a human or mammalian body fluid such as a sample chosen from a mammalian body fluid such as mucus, including nasal or laryngeal mucus, sputum, saliva, tears, feces, including fecal extracts, urine, a biopsy, including homogenized biopsies or extracts thereof, whole blood or a blood derived sample like blood plasma or blood serum.

When a blood derived sample is used, the sample will generally be cell depleted, typically close to 100% depletion, but less depletion can also be used when preferred. Plasma samples can also be prepared by adding materials that prevent or delay coagulation, such as EDTA, citrate, heparin or the like.

The subject from which the sample is taken will most often be a human or a non-human animal subject, preferably a human, a canine or feline mammal. Most preferably the subject is a human subject. The subject may be a subject with or without existing clinical manifestations of viral disease, ongoing or previous.

The assay is homogenous, meaning that the assay reaction takes place and the signal is generated as well as detected in a liquid phase. Thus, at no point in the homogeneous assay methods disclosed herein, no anti-viral component of the sample or any complex comprising antiviral antibody material, is/are separated from the bulk liquid phase by binding or capture onto a solid support. There is therefore no need for any step of phase separation, which significantly simplifies the process. Pre-concentration of the anti-viral antibodies from the sample to be analyzed can be performed, but this constitutes a less preferred embodiment, since it increases the number of assay steps.

As used herein, the term “capture molecule” is used to indicate a binder, partner, antigen or ligand which binds specifically to a component of interest, here an anti-virus antibody. Such capture molecules will generally show little or no binding affinity for other components of the sample, such as other peptides, proteins or antibodies. For example, the affinity for non-anti-SARS-CoV-2 antibodies from the same subject should be no more than 1/100th, preferably no more than 1/1,000th, and most preferably no more than 1/10,000th of the affinity of the capture molecule or ligand for anti-SARS-CoV-2 antibodies. Importantly the capture molecule should not exhibit any or only negligible cross-reactivity against antibodies against other normally circulating viruses if the assay is specific for corona viruses. Correspondingly, if the assay is specific for an individual corona virus, such as the SARS-CoV-2 virus, the capture molecule should not exhibit any or only negligible cross-reactivity against antibodies against other corona viruses, such as seasonal influenza.

The expression “steric hindrance” is used to describe a situation where the spatial structure of a molecule prevents or slows down inter-molecule interaction, for example the binding of an antibody to an antigen.

According to a first aspect, the present disclosure makes available a method for detection of an antibody against a virus in a sample suspected of comprising antibodies against said virus, comprising:
contacting the sample with a nanoparticle having capture molecules and co-molecules immobilized thereon, wherein a capture molecule comprises a peptide that is specifically recognized by the antibody and wherein a co-molecule is not specifically recognized by the antibody and has a molecular weight smaller than the molecular weight of the capture molecule,
forming a complex of the antibody with the capture molecule immobilized on the nanoparticle in the presence of the antibodies in the sample, and
determining a signal indicative for the presence of the complex.

According to an embodiment, said capture molecule is an epitope isolated from a spike, envelope, membrane or nucleocapsid protein of said virus.

The method is applicable to any virus, but according to an embodiment currently preferred by the inventors, said virus is a SARS-CoV-2 virus including current and future variants thereof.

According to an embodiment of the method, the co-molecules when immobilized on said nanoparticle separate the capture molecules so that an average distance between two adjacent capture molecules is greater than a distance between antigen binding sites of the anti-virus antibody to be detected.

The distance between the antigen binding sites of an antibody can be determined for example using neutron and X-ray scattering, as disclosed for example in Sosnick et al., Distances between Antigen-binding Sites of Three Murine Antibody Subclasses Measured Using Neutron and X-ray Scattering, in Biochemistry, 1992, 31, 1779-1786.

According to another embodiment of the first aspect and freely combinable with any embodiments thereof, the co-molecule is a molecule having at least one primary amine moiety. Amines are classified according to the number of carbon atoms bonded directly to the nitrogen atom. Thus, a primary amine has one alkyl (or aryl) group on the nitrogen atom, a secondary amine has two, and a tertiary amine has three. Preferably the co-molecule is chosen from the group comprising hydroxylamine, tris(hydroxymethyl)aminomethane, amino acids, low molecular weight peptides and ethanolamine. According to a preferred embodiment, the co-molecule is ethanolamine.

According to another embodiment, the capture molecule is an epitope isolated from a spike protein of a SARS-CoV-2 virus including variants thereof. The co-molecule then preferably has a molecular weight in the range of 50-1500 Da. For future virus variants, and different capture molecules, the molecular weight of the co-molecule can be adjusted accordingly.

According to an alternative embodiment of the first aspect, the co-molecule is low-molecule weight peptide, preferably a peptide having a molecular weight in the range of 300-1500 Da. For future virus variants, and different capture molecules, the molecular weight of the co-molecule can be adjusted accordingly.

According to an embodiment of the first aspect freely combinable with any embodiments herein, the signal generated by said complex is measured by turbidimetry or nephelometry, and the particles used in said method have a median diameter preferably less than 400 nm, such as a diameter in the interval of 10 to 300 nm and more preferably a diameter in the interval of 100 and 250 nanometer, most preferably 100-150 nanometer.

According to another embodiment of the first aspect freely combinable with any embodiments herein, the sample is a sample chosen from a mammalian body fluid such as mucus, including nasal and laryngeal mucus, sputum, saliva, tears, feces, including fecal extracts, urine, biopsies, including homogenates and/or extracts of biopsies, whole blood or a blood derived sample like blood plasma or blood serum. According to a preferred embodiment, the sample is a sample chosen from mammalian blood plasma and serum.

According to an embodiment of the first aspect and freely combinable with any embodiments mentioned herein, the method further comprises a calibration step using calibration samples having an antibody content ranging from 0 to 100 μg/ml of anti-SARS-CoV-2 IgG antibody in plasma, for example 0 to 20 μg/ml. When IgM is determined, the method further comprises a calibration step using calibration samples having an antibody content ranging from 0 to 200 μg/ml of anti-SARS-CoV-2 IgM antibody in plasma. For IgA, the corresponding calibration range may be 0-100 μg/ml in plasma.

According to an embodiment of the above first aspect, said antibodies are antibodies specific to a virus belonging to the corona virus family, for example but not limited to the currently known human coronavirus types 229E (alpha coronavirus), NL63 (alpha coronavirus), 0043 (beta coronavirus), HKU1 (beta coronavirus), MERS-CoV (a beta coronavirus that causes the Middle East Respiratory Syndrome, MERS), SARS-CoV (a beta coronavirus that causes severe acute respiratory syndrome, SARS), and SARS-CoV-2, the novel coronavirus that causes coronavirus disease 2019, COVID-19.

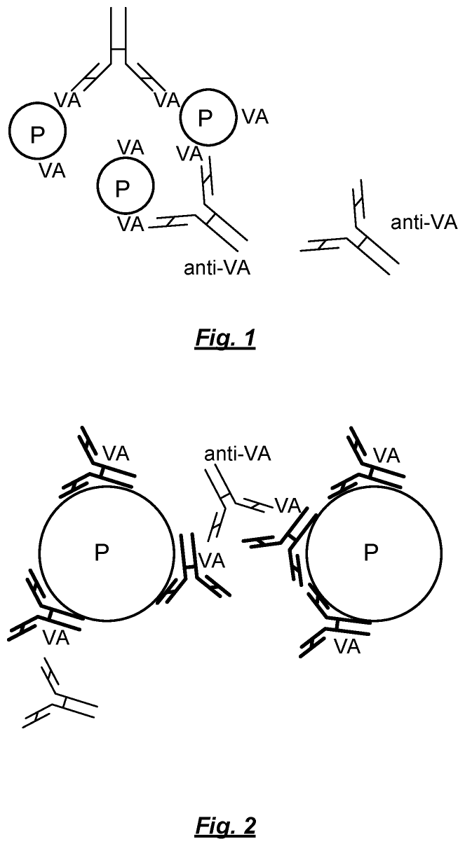

Preferably said antibodies are antibodies directed against an epitope isolated from the virus spike, envelope, membrane or nucleocapsid proteins. It is however contemplated that other viral proteins or peptides are identified, and in the following description, all potential antigens are referred to as “viral antigens”, abbreviated “VA” in the figures. Different principles of binding and subsequent aggregation are shown in FIGS. 1, 2 and 3. Problems with conventional assays are shown in FIGS. 4A and 4B, whereas an embodiment of the present disclosure is illustrated in FIG. 5.

According to a preferred embodiment, said antibodies are antibodies specific to SARS-CoV-2. Preferably said antibodies are antibodies directed against a conserved epitope from SARS-CoV-2, for example a conserved epitope isolated from the SARS-CoV-2 spike protein, for example the epitope referred to as “S1” but other naturally occurring or recombinant viral antigens are also contemplated.

According to an embodiment freely combinable with all the above embodiments, said capture molecule is bound to at least one signal generating moiety. Signal generating moieties can be organic or inorganic particles, for example monodisperse polymeric particles, such as latex particles, or metal particles, such as colloidal gold. Methods and reagents for coupling peptide or protein antigens to particles are known to persons skilled in the art.

A preferred method is direct covalent coupling of the capture molecule to a surface functionalized latex particle. There are several commercially available surface modifications for latex particles. For protein conjugation, chloromethyl functional groups, carboxyl functional groups, or N-hydroxy-succinimide (NHS) functional groups are most commonly used. The functional groups react with primary amine groups in the capture molecule. Chloromethyl groups and N-hydroxy-succinimide (NHS) functional groups are reactive towards amine groups without using an activation step and are preferred modes and means of surface functionalization. The result of the coating is controlled by physical and chemical parameters of the coating reaction and by introduction of a co-binding protein or co-binding molecule with functional groups that are reactive to the functional groups on the particle surface.

Another method is that based on the biotin-avidin, biotin-Streptavidin or biotin-NeutrAvidin binding reaction. An antigen is conjugated with biotin and brought in contact with particles that have been activated, for example coated with avidin or other biotin-binding proteins, including Streptavidin or NeutrAvidin. By controlling the physical and chemical parameters of the coating reaction, as well as the ratio of viral antigen to particles, the result of the coating can be adjusted as desired.

Yet another type of generating particles are different biopolymers, in particular carbohydrate-based particles, such as but not limited to dextran and similar. Commercial dextran products are on the market, for example under the trademark Sephadex® available as beads in different size ranges generally within a range of 20-150 μm. Other insoluble carbohydrate monodisperse particles are also contemplated.

The particles, both in the “nude” and coated state, preferably have a diameter which does not itself cause absorption of the wavelength of light used for spectrophotometric determination.

Most preferred are particles which may be suspended in aqueous media, and which are smaller than the wavelengths of red, preferably blue, light and most preferably less than 400 nm in diameter, such as a diameter in the interval of 10 to 300 nm and more preferably particles with a median diameter in the interval of 100 and 250 nanometer, more preferably 100-150 nanometer.

Latex particles, also called beads, are available in a wide range of sizes, from approximately 20 nm to 15 μm, and in a wide range of surface functional types. For example, chloromethyl functionalized, N-hydroxy-succinimide (NHS) functionalized, or carboxyl charge-stabilized hydrophobic latex beads can be used for physical adsorption of antigens or antibodies, or for covalent coupling of components to the particles. Another type, sulfate latex beads are appropriate for immunoassays that rely upon physical adsorption of antigens or antibodies. Beads of different size and surface chemistry are available from several suppliers, for example from ThermoFisher Scientific, Eugene, Oregon, USA.

The particles may also be made of glass, silica latex, metal (e.g. gold) polymeric material (e.g. polyethylene). The particles to which the specific capture molecule, e.g. virus material, viral antigens, viral conjugates and antibodies binding to viral material, are bound, are typically spherical. The size of the particles used in the assay may influence the precision of the assay, with which the anti-virus antibodies in the sample material are measured. Larger particles allow for measurement of lower concentrations of the analytes to be measured, while smaller particles allow for higher binding capacity but may often lead to reduced sensitivity. For example, doubling the particle diameter can reduce the binding capacity of a mass unit of particles by half.

Additionally, increasing the particle diameter increases the level of the background light absorbance and light scattering at the wavelengths typically used in such assays, e.g. 330 to 600 nm.

The size of the particles used to bind the virus antigen material to be reacted with antibody in the sample material, can be optimized depending on the size of the viral antigen material directly or indirectly bound to the nanoparticles to which the antigenic material is coupled. The size of particles is preferably balanced to consider stability, in addition to sensitivity and binding capacity. The reagents need to be stable, for example have a shelf life in the range of months to about a year, preferably at least 1 year in unopened refrigerated packaging.

Monodisperse particles are more preferred, and without wishing to be bound by theory, it is believed that by using a solid support or matrix (e.g. nanoparticles) which is substantially all of the same size, i.e. monodispersed, it may be that the sensitivity of the turbidimetry assay is increased.

Preferably a detectable signal is generated by the formation of a complex comprising said signal generating moiety and at least one other signal generating moiety of the same or different type. Preferably said signal generating moiety is a nanoparticle.

According to an embodiment freely combinable with all the above embodiments, the binding of the homogeneous particles carrying the specific capture molecule and anti-virus antibody react to generate a signal detectable by turbidimetry or nephelometry. The principles of such measurements are outlined for example in the chapter “Nephelometry and turbidimetry” in the textbook Laboratory Instrumentation, by Mary C. Hagen et al., John Wiley & Sons, 1994.

The turbidimetric or nephelometric methods according to any embodiment above may additionally comprise a step of adding auxiliary reagents, for example preservatives, stabilizers and opacity enhancers, for example by including such reagents or reagent such as an opacity enhancer, in one or more of the reagents used in the method. Examples of opacity enhancers include different qualities of polyethylene glycol (PEG). Interestingly, PEG may also act as a potentiating agent enhancing the interaction between antibodies and viral antigens. Different PEG-derivates can also be used for increasing solubility, improve stability, and for example reduce or prevent non-specific aggregation.

PEG is available in a number of molecular weights. The most common size for use in turbidimetric assays is PEG 6000. When determining how to incorporate the polymer into the assay, one needs to consider that increasing the PEG concentration will increase the apparent rate of agglutination. In the case of a turbidimetric assay system, this may also increase the signal change. Further, there is a close link between protein loading on a particle and PEG concentration. As the protein loading increases, the sensitivity to PEG increases. In other words, less PEG is required to achieve an increase in the apparent rate of agglutination. Above a critical concentration of PEG at a given protein loading, the protein loaded particles will agglutinate in the presence of any other protein, increasing problems of nonspecific aggregation. Microsphere auto-agglutination can also increase.

Another aspect of the disclosure is a kit for the detection of antibodies against a virus in a sample, for example performing a method according to the first aspect and any embodiments thereof. Thus, the present disclosure makes available a kit for detection of antibodies against a virus in a sample, comprising at least one specific capture molecule for an anti-virus antibody and a co-molecule immobilized to a nanoparticle, wherein two or more such capture molecules are immobilized on each particle, wherein
said capture molecule is an epitope isolated from a spike, envelope, membrane or nucleocapsid protein of said virus,
said co-molecule is not specifically recognized by the antibody and has a molecular weight smaller than that of said capture molecule,
said co-molecules when immobilized on said nanoparticle separate the capture molecules so that an average distance between two adjacent capture molecules is greater than a distance between the antigen binding sites of the anti-virus antibody to be detected.

According to an embodiment of said second aspect, the capture molecule is an epitope isolated from a spike protein of a SARS-CoV-2 virus or variants thereof.

According to another embodiment of said second aspect, freely combinable with the above, the co-molecule has a primary amine moiety.

Preferably the co-molecule is chosen from the group comprising hydroxylamine, tris(hydroxymethyl)aminomethane, amino acids, low molecular weight peptides and ethanolamine. According to a preferred embodiment, the co-molecule is ethanolamine.

According to an embodiment of said second aspect, the co-molecule has a molecular weight in the range of 50-1500 Da. For future virus variants, and different capture molecules, the molecular weight of the co-molecule can be adjusted accordingly.

According to an alternative embodiment of the second aspect, the co-molecule is a low-molecular weight peptide, preferably a peptide having a molecular weight in the range of 300-1500 Da. For future virus variants, and different capture molecules, the molecular weight of the co-molecule can be adjusted accordingly.

According to another embodiment of the second aspect and freely combinable with any embodiments mentioned herein, the kit further comprises a set of calibrators having an antibody content ranging from 0-100 μg/ml, preferably 0 to 20 μg/ml of anti-SARS-CoV-2 antibody (IgG) in plasma.

According to another embodiment of the second aspect and freely combinable with any embodiments mentioned herein, the kit further comprises a set of calibrators having antibody contents ranging from 0 to 100 μg/ml of anti-SARS-CoV-2 antibody (IgG) in plasma. When the method is adapted to quantify IgM, the same or a different clinical range will be used, for example 0-200 μg/ml IgM, and for IgA, 0-100 μg/ml is contemplated.

In a kit according to the second aspect or any embodiment thereof, the particles used in said kit have a diameter preferably less than 400 nm in diameter, such as a diameter in the interval of 10 to 300 nm and more preferably a median diameter in the interval of 100 and 250 nanometer, most preferably 100-150 nanometer.

A kit according to the second aspect or any embodiment thereof may optionally include auxiliary reagents for performing the measurement, for example an opacity enhancer.

A third aspect is a nanoparticle for use in a method according to the first aspect or any one of embodiments thereof, or for incorporation as a component in a kit according to the second aspect or any one of the embodiments thereof, comprising a capture molecule chosen from an epitope isolated from a spike, envelope, membrane or nucleocapsid protein of a virus, and a co-molecule immobilized to said nanoparticle, wherein said capture molecule is not specifically recognized by the antibody and has a molecular weight smaller than that of said capture molecule, and wherein said co-molecules when immobilized on said particles separate the capture molecules so that an average distance between two adjacent capture molecules is greater than a distance between the antigen binding sites of the anti-virus antibody to be detected.

Preferably the capture molecule is an epitope isolated from a spike protein of a SARS-CoV-2 virus or variants thereof.

According to an embodiment, said co-molecule molecule has a primary amine moiety, and said co-molecule is preferably chosen from the group comprising hydroxylamine, tris(hydroxymethyl)aminomethane, amino acids, low molecular weight peptides and ethanolamine. Most preferably the co-molecule is ethanolamine.

According to an embodiment of said third aspect, the co-molecule has a molecular weight in the range of 50-1500 Da. For future virus variants, and different capture molecules, the molecular weight of the co-molecule can be adjusted accordingly.

According to an alternative embodiment, the co-molecule is a low-molecular weight peptide, preferably a peptide having a molecular weight in the range of 300-1500 Da. For future virus variants, and different capture molecules, the molecular weight of the co-molecule can be adjusted accordingly.

According to an embodiment of said third aspect, the nanoparticles have a median diameter preferably less than 400 nm, such as a diameter in the interval of 10 to 300 nm and more preferably a diameter in the interval of 100 and 250 nanometer, most preferably 100-150 nanometer.

A fourth aspect of the invention is a reagent solution comprising a nanoparticle according to the third aspect and any embodiment thereof. Such reagent solution may further include functional additives and further reagents, such as a preservative, pH-regulating buffers, density regulating agents, stabilizers, and one or more opacity increasing agents, for example polyethylene glycol (PEG).

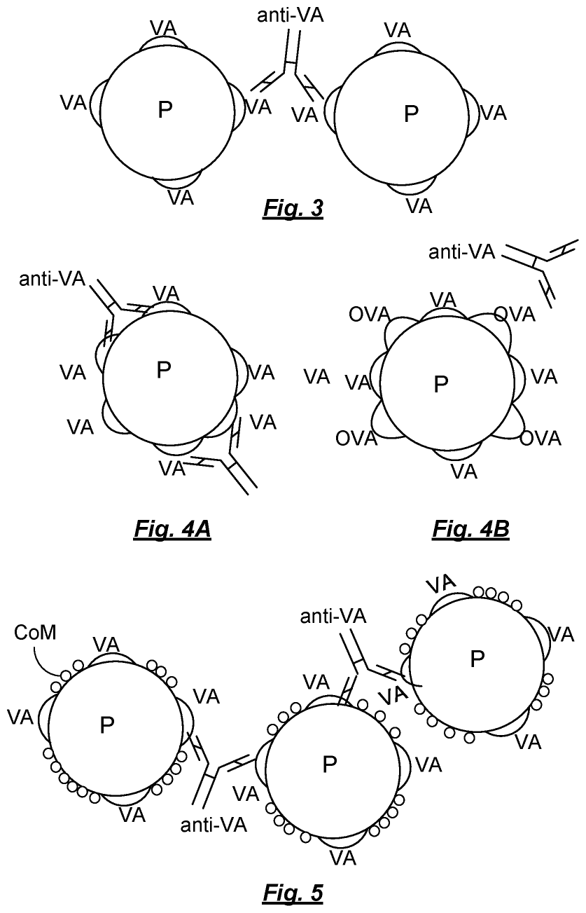

FIG. 3 shows how a suitable spacing of the virus antigens allows an anti-VA antibody to bind to virus antigens (VA) on two particles (P), resulting in a measurable aggregation. However, as illustrated in FIG. 4A, if the virus antigen (VA) is present too densely on the particles (P), it is possible that one or more antibodies bind to multiple virus antigens on the same particle, which doesn't result in the desired aggregation of particles.

FIG. 4B illustrates a situation where a co-molecule is used to distribute the virus antigen (VA) evenly on the particles (P), but where said co-molecule is equal or larger in size, thus “over-shadowing” the antigen, creating steric hindrance and preventing or reducing binding between the antibody and the antigen. Ovalbumin (OVA) is here used as an example of such, frequently used co-molecule, which the present inventors however found to be unsuitable.

FIG. 5 shows schematically a scenario according to the present disclosure, where a co-molecule (CoM) is chosen so that it distributes the antigen (VA) over the surface of the particles (P), at distance between antigen that is larger than the distance between the arms of the antibody in question (anti-VA). At the same time, the co-molecule is of a size/molecular weight which is smaller in relation to the antigen, so that the co-molecule does not create steric hindrance.

As the various signal generating moieties are brought together by the act of binding, and particularly by the act of dual binding and cross-linking, by the antibodies in the sample material specifically reacting with the viral antigens, whole or fragmented, a measurable aggregation occurs, the degree of which can be correlated to the concentration of antibodies in the sample.

In turbidimetry, it may be of great value to bring together several signal generating moieties together in greater meshwork, and especially increase the absorption of light absorption or the spreading of light. Most often, the spread of light and absorption of light increases with increasing particle size and/or increasing size of meshwork of materials.

In a turbidimetric assay, opacity may be generated by contacting anti-viral antibody material in the sample with a viral antigen—whole or particle or fragment or conjugated or derivatized, preferentially bound to a signal generating moiety, such as a nanoparticle. Additionally, one or more opacity increasing agents can be used, for example polyethylene glycol (PEG).

Typically, calibration samples having anti-viral antibodies with affinity for virus antigens, virus particles, virus fragments and/or virus conjugates, will be used to calibrate and standardize the assay methods according to the present invention, often prepared from well characterized patient samples.

The primary goal for the method disclosed herein is to qualitatively, quantitatively or semi-quantitatively determine the presence of antibodies reactive towards viruses and/or analogs or derivatives or fragments of viruses in a sample to be tested. This will provide an indication or a measure of the risk for related previous or ongoing virus infections in a person from which a sample has been taken. Such information is important when diagnosing ongoing infections as well as determining the degree of immunity in a population.

The herein disclosed method for the assessment or quantitation of antibodies in a sample reactive to the viral antigen or fragments or derivatives bound to the particulate material in the assay mixture can be used to generate turbidimetric or nephelometric signals to diagnose or monitor viral infection, acquired immunity in the aftermath of an infection, or the effect of vaccines.

The turbidimetric or nephelometric signal formed in the assay mixture as a result of the aggregation of particles, is then used to determine the level of said antibodies reactive to the virus and/or analogs or derivative in the sample material, and correlating the thus determined level of turbidimetric or nephelometric signal with the existence of said antibodies and the risk for ongoing or previous infection or disease in the subject from which the sample has been generated. These signals of turbidimetry or nephelometry are then often correlated to non-existence or presence or even more increased levels of antibodies towards virus materials, fragments or derivatives conjugated to particle material.

It is a significant advantage that the assays disclosed herein are homogenous, or use suspensions of particles, as this provides considerable benefits in simplifying the method, making it easier to automate, reducing the number of reagents and making it possible to avoid separation steps or washing steps to remove unbound reagents, which otherwise constitute a considerable challenge when designing an assay.

In this context and in the assay as disclosed herein, the term “homogeneous assay” here denotes an assay method in which the sample and at least one reagent are mixed, a signal is generated, and that signal detected without any separation or washing steps involving phase separation.

Similarly, “homogenous” is used to underline that the assay mixture is in a stable fluid form. Although the mixture or assay mixture can comprise particles, these are stable in a single fluid or kept in a suspension in a single liquid fluid. This is optionally achieved or facilitated by regulating the density or the specific weight of the mixture to keep particles in suspension without settling.

Most preferred are suspensions that do not settle during the time needed to perform the experiments, and also preferred are suspension that can be stored for weeks—and even months—without settling.

Binding or immobilizing the virus antigen to the particles may be achieved using any conventional technique. A preferred method is direct covalent coupling of the capture molecule to a surface functionalized latex particle. There are several commercially available surface modifications for latex particles which are reactive to e.g., proteins. A preferred surface modification is chloromethyl activated polystyrene nanoparticles (available from ThermoFisher Scientific, USA). The antigen may be immobilized to the latex particles by agitation in buffer (e.g. at room temperature for 4-24 hours). The functional chloromethyl groups react with primary amine groups in the antigen without using an activation step. The result of the coating is controlled by physical and chemical parameters of the coating reaction and by introduction of a co-binding protein or co-binding molecule with functional groups that are reactive to the functional groups on the particle surface. The size of said co-molecule is—according to the present disclosure—chosen such that it is smaller/has a lower molecular weight than the capture molecule, so that it serves to distribute the capture molecule evenly on the particle surface, creating a suitable distance between biding partners, without interfering with the interaction of the capture molecule and anti-virus antibody.

As another example, avidin or neutravidin (available e.g. from ThermoFisher Scientific, USA) may be immobilized on chloromethyl activated polystyrene nanoparticles (also from ThermoFisher Scientific, USA) by agitation in buffer (e. g. at room temperature for 24 hours) and then used in conjunction with biotin labelled virus peptide (prepared according to conventional techniques known to a skilled person). Thus, for example, plasma taken from the subject to be tested is added to a solution of avidin- or neutravidin-coated nanoparticles in a quartz cuvette of a spectrophotometer, followed by the addition of biotin labelled virus peptides.

Alternatively, the biotin labelled peptide may be added prior to the addition of plasma or serum. In other words, whilst the same reagents are typically used regardless of the instrument used for turbidity detection, the precise sequence in which the various reagents are added may vary. Generally, the sequence used should be in accordance with the instructions accompanying the spectrophotometer used (e.g. a Shimadzu UV-160 spectrophotometer).

In an alternative experimental set-up, binding or immobilization of the virus antigen to a particulate signal generating moiety may be achieved using any other conventional technique. For example, non-specific IgG or hydrophobic proteins may be immobilized on chloromethyl activated polystyrene nanoparticles (available from ThermoFisher Scientific, USA) by agitation in buffer (e.g. at room temperature for 24 hours) and coupled to a virus peptide using cross-linking reagents (available from ThermoFisher Scientific, USA) prepared according to conventional techniques in the art) prior to or after binding of the IgG molecules to the nanoparticles. Thus, for example, plasma taken from the subject to be tested for potential for, or propensity to, virus infection is added to a solution of coated nanoparticles in a quartz cuvette of a spectrophotometer. Turbidimetric readings are then taken.

Turbidimetric readings are made (i.e. the light absorption or light scattering at a suitable wavelength is measured at regular intervals) and the measured value relative to a reference is determined. Analogous methods are used for other detection methods, as is known in the art. Optionally, multiple wavelength instruments may be used to make turbidimetric readings and may provide more precise results. Suitable instruments for taking turbidimetric readings include but are not limited to for example the Cobas c501 or c701 from Roche; the Architect instrument range from Abbott; and AU480 and AU680 instruments from Beckman Coulter.

The same reagents are typically used regardless of the instrument used for turbidity detection, but the precise sequence in which the various reagents are added may vary. Generally, the sequence used should be in accordance with the instructions accompanying the clinical analyzer used.

Various features of the assay methods of the invention may be optimized to improve the results. These include the following preferable features, which are all freely combinable and may be used independently.

Optionally, in determining anti-viral antibody concentration, a kinetic reading mode may be used. This method may preferably be used in all aspects of the invention, particularly when using the turbidimetry technique.

In general, in addition to the sample under evaluation, calibration samples with known anti-viral antibody contents will also be assessed in the performance of the assay method. Such determinations can be used to establish a calibration curve or calibration algorithm from which the anti-viral antibody content of the sample under evaluation can be determined.

Preferably calibration samples having anti-viral antibody contents of up to 100 μg/ml (e.g. any selection of 0, 1, 2, 3, 4, 5, 6, 7, 8, 9, 10, 20, 40, 60, 80, and 100 μg/ml) will be used, preferably calibrators in a range of 0-20 μg/ml for IgG. Similar concentration ranges are contemplated also for IgM and IgA. The concentration of antibody in the calibration samples is achieved by preparing a standard solution of antibody in a suitable medium, confirming the concentration by titration, and preparing the necessary standards by suitable dilution. The calibration ranges are chosen with consideration of the clinical ranges observed for a population having undergone vaccination, patients suffering from an active infection, as well as patients having recovered from an infection. In the case of recurrent, known viral infections, the clinical ranges may be known from literature, or easily determined, but in the case of previously unknown viruses, an understanding of clinical ranges will build up over time. In acute situations, such as a pandemic, clinical ranges can be approximated based on historical data and preliminary assessment och recovering patients.

In the case of SARS-Cov-2 there are early indications that IgA and IgM peaked at 8.84 μg/ml and 7.25 μg/ml respectively, and IgG peaked at 16.47 μg/ml (Huan Ma et al., COVID-19 diagnosis and study of serum SARS-CoV-2 specific IgA, IgM and IgG by chemiluminescence immunoanalysis, MedRcIV Preprint posted Apr. 30, 2020, doi: https://doi.org/10.1101/2020.04.17.20064907)

Various types of known assay formats may be used to perform a homogeneous assay, and those in which a signal is generated by bringing components of sample together are of particular value. Antibodies have multivalent binding sites, which allows them to serve as crosslinkers. This enables the formation of larger aggregates resulting in a detectable signal.

A significant advantage of a homogenous assay is that homogeneous assays are easily automatized, at least when compared to assay methods based of separation- and washing steps. Therefore, simpler and faster instruments can be used, and both the purchasing, and the maintenance of the instruments becomes less costly. High throughput of samples becomes easier and the running costs lower.

Another advantage is that the methods disclosed herein are applicable to being performed on open platforms i.e. the turbidimetric or nephelometric methods can be performed on any commercially available robot or automated platform, for example a classical trimetric instrument like Hitachi 711 or the Mindray BS200 instrument, or more modern instruments such as Roche Cobas c501, c502, c702, the Beckmann Coulter AU480, DxC 700 AU, AU 5800, Abbott Architect c4000, or Mindray BS-380 and BS-400.

The operating principle of these instruments can be described in a simplified fashion as “mix sample and reagents and measure”. The present inventors estimate that there are more than 430.000 open platform instruments available globally, in hospitals and central laboratories, very well suited for mass testing of patient samples. The present inventors have surprisingly realized that the method disclosed herein enables straight-forward “mix sample and reagents and measure” instrument measurements of antibodies towards virus and analogues. Thus, a more effective clinical set of assays is enabled.

Another advantage is the flexibility of the method. By changing the capture molecule to correspond to potential mutation variants of the virus, the method can be used to detect and monitor the presence of antibodies also against future virus variants.

Aspects and embodiments will now be illustrated by the following non-limiting examples.

### EXAMPLES

### Example 1. Preparation and Evaluation of Particle Model Systems for Turbidimetric Antibody Detection

The purpose of this investigation was to prepare a model assay for antibody detection and evaluate a low molecular weight substance (ethanolamine) instead of a protein (ovalbumin) as co-molecule in the coating process. Particles were coated with anti-human IgG and IgG was detected as the analyte in an automated chemical analyzer (Mindray BS-240). For antibody detection, steric hindrance for the target antibodies in serum must be considered in the coating procedure. The inventors contemplated that the use of a smaller co-molecule would expose more of the attached ligand to the antibody analyte. Therefore, smaller co-molecules, i.e. co-molecules having a lower molecular weight than the capture molecule, were tested and compared to a commonly used co-protein, ovalbumin. In this example ethanolamine, having a molecular weight of 61 g/mol, was used as an alternative to ovalbumin with a molecular weight of 45000 g/mol.

### 1.1 Reagents

In this example, the following material and reagents were used:

TABLE 1
Material and reagents used in Example 1

| | |
|---|---|
| Particles | 130 nm chloromethyl particles, 4% (w/v), 40 |
| | mg/ml (ThermoFisher Scientific) |
| Antibody | Goat anti-human IgG, 35 mg/ml, lot 99994 |
| | (109-001-006, Jackson ImmunoResearch) |
| Co-molecule 1 | ethanolamine 1 mM and 10 mM in coupling |
| | buffer |
| Co-molecule 2 | Ovalbumin |
| Coupling buffer: | Low salt concentration neutral phosphate buffer |
| Quenching buffer | Ovalbumin (0.5 g/L) in a neutral phosphate |
| | buffer with surfactants |
| Storage buffer | Ovalbumin (0.5 g/L) in a neutral phosphate |
| | buffer with surfactants |

### 1.2 Coating Procedure

Three different particle batches were prepared. In all batches 50% surface coverage of the antigen were used. Surface area and coverage was calculated according to the Bang TechNote No. 205 “Covalent Coupling”, March 2013, assuming a C value of approximately 3 mg/m², as for BSA. This method was first disclosed in Cantarero et al., The adsorptive characteristics of proteins for polystyrene and their significance in solid phase immunoassays, in Anal Biochem, 105, 375-382 (1980).

For 130 nm particles and anti-IgG this corresponds to a particle:protein ratio of about 16:1 which was used for all batches. In one of the batches, ovalbumin was added as a co-protein, in an equal amount (mg) as the anti-IgG. In the other two batches, ovalbumin was replaced by a small molecule including a primary amine moiety, here ethanolamine. Ethanolamine was added in a 1:1 molar ratio and a 19:1 molar ratio. The 19:1 ratio was selected to represent a 1:1 molar ratio of ethanolamine:primary amine moieties in the protein.

The particles were washed two times using 10 times the particle volume of coupling buffer (see Table 1). The suspension was centrifuged at approx. 13000*g for 20 min in between each washing step.

After the second wash, the particles were resuspended in 5 times the particle volume in said coupling buffer. The particles were completely resuspended by vortexing and sonication. Anti-human IgG and ovalbumin or ethanolamine were dissolved in 5 times the particle volume of coupling buffer. The particle suspension and protein solution were combined and allowed to react at room temperature (18-25° C.) over night with constant mixing.

After the conjugation, the reaction mix was centrifuged at 13 000*g for 20 minutes and the buffer was changed into quenching buffer (See Table 1). Quenching was performed at constant mixing for 2.5 hours. Finally, the coated particles were washed twice in storage buffer (See Table 1) and diluted to OD=5 at 546 nm.

### 1.3 Evaluation of Performance

In order to evaluate the performance of the particles, IgG samples were prepared as follows: Human plasma (stored at −20° C.) was diluted in 1× Blocker BSA in TBS (RF234219, ThermoFisher) to prepare a stock solution used to prepare a range of samples having different concentrations. The IgG concentration in human plasma was assumed to be 10.6 mg/ml based on the reference range 6.7-14.5 mg/ml (Huan Ma et al., Supra). By diluting the stock solution, 9 different concentrations were obtained, as shown in Table 2 below. These were then analyzed in an automated chemical analyzer (Mindray BS-240).

TABLE 2
Dilution of IgG samples

| Dilution | Approx. IgG conc (μg/mL) |
|---|---|
| 1:12800 | 0.83 |
| 1:6400 | 1.66 |
| 1:3200 | 3.31 |
| 1:1600 | 6.66 |
| 1:800 | 13.3 |
| 1:400 | 26.5 |
| 1:200 | 53.0 |
| 1:100 | 106 |
| 1:50 | 212 |

These samples were tested in the automated chemical analyzer using the method settings shown in the table below:

TABLE 3
Instrument settings used in the evaluation

| | |
|---|---|
| Instrument: | Mindray BS-240 |
| Sample type | Serum |
| Chemistry | IgG in serum |
| Reaction type | Endpoint |
| Reaction direction | Positive |
| Primary wavelength (nm) | 546 |
| Secondary wavelength | None |
| Unit | RLU |
| Decimal | 0.1 |
| Blank time (s) | 2 and 2 |
| Incubation time (s) | 21 |
| Reaction time (s) | 3 and 25 |

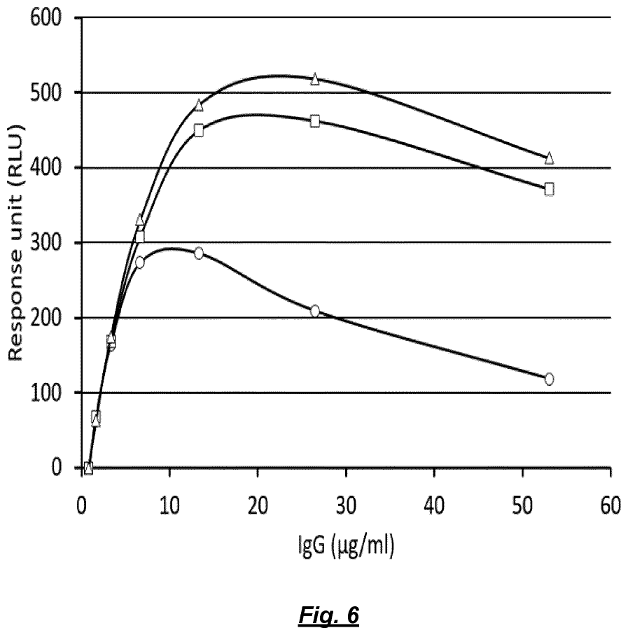

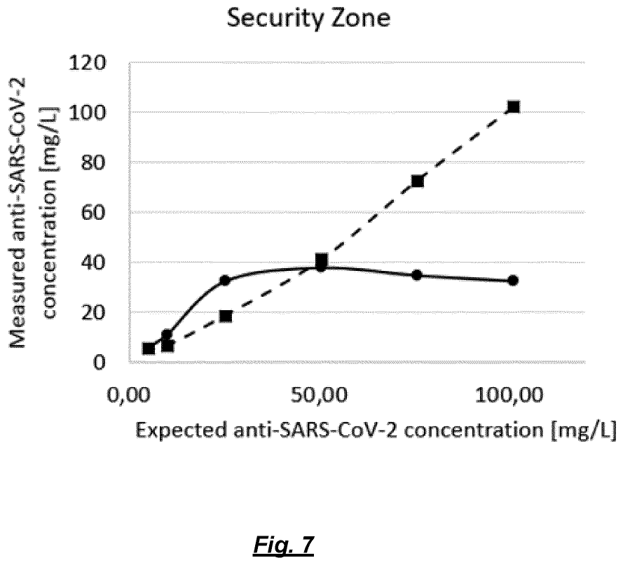

The tests were run using a sample size of 7 μl (IgG solution), 105 μl reaction buffer (a physiological buffer with Tween 20 and preservative) and 17.5 μl particle solution OD=5. The results for the three different baches measured as relative response in RU are presented in FIG. 6. The responses are adjusted to show 0 RU for the lowest concentration for comparison. Results are shown for concentrations ranging from 0-53 μg/ml.

It is evident that exchanging the co-protein ovalbumin with a low molecular weight molecule containing a primary amine moiety, here exemplified by ethanolamine, greatly improves the turbidimetric signal vs concentration, and extends the measurement range for the analyte (i.e., later hook). The inventors found that the co-molecule helps to distribute the antigen evenly over the surface of the particles, and that the small size of the co-molecule does not induce steric hindrance. The results also clearly indicated that the inventors have been successful in conjugating the antigen to the latex particles.

### Example 2. Preparation of Particles Coated with SARS-Cov-2 S1 Subunits

The purpose of this example was to test the feasibility of coupling a virus epitope to particles, and to evaluate said particles in an automated chemical analyzer (Mindray BS-240). Based on the results of Example 1, ethanolamine was chosen as the co-molecule coupling the virus epitope to the particles.

### 2.1 Reagents

In this example, the following material and reagents were used:

TABLE 4
Material and reagents used in Example 2

| | |
|---|---|
| Particles | Chloromethyl latex, 4% w/v, 0.1 μm |
| | (approx 110-140 nm) (C47161, Thermo |
| | Fisher Scientific) |
| Co-molecule | ethanolamine 10 mM in coupling buffer |
| Virus epitope | Recombinant SARS-CoV-2 S1 (710091, |
| | Medix Biochemica) |
| Coupling buffer | Low salt concentration borate buffer, pH 9 |
| Quenching buffer | Ovalbumin (0.5 g/L) in a neutral |
| | phosphate buffer with surfactants |
| Storage buffer | Ovalbumin (0.5 g/L) in a neutral |
| | phosphate buffer with surfactants |

### 2.2 Coating Procedure

SARS-COV2 S1 coated particles were prepared according to a modified version of the protocol used for producing the model particles for antibody detection using anti-IgG. Ovalbumin was replaced with ethanolamine in a 19:1 molar ratio ethanolamine:S1.

120 nm particles (16 mg particles/mg antigen) were washed two times using 10 times the particle volume of coupling buffer (See Table 4). The suspension was centrifuged at approx. 13000*g for 20 min in between each washing step.

After the second wash, the particles were resuspended in 5 times the particle volume in coupling buffer. The particles were completely resuspended by vortexing and sonication. Recombinant S1 subunit (product no 710091, Medix Biochemica) and ethanolamine (76 μl of the 10 mM ethanolamine in coupling buffer per mg antigen) were dissolved in 5 times the particle volume of coupling buffer. The particle suspension and protein solution were combined and allowed to react at room temperature (18-25° C.) over night with constant mixing.

After the conjugation, the reaction mix was centrifuged at 13 000*g for 20 minutes and the buffer was changed into quenching buffer (See Table 4). Quenching was performed at constant mixing for at least 30 minutes. Finally, the coated particles were washed at least twice in storage buffer (See Table 4) and diluted to OD=5 at 546 nm.

### Example 3. Evaluation on Mindray BS-240

The inventors had access to a range of turbidimetric instruments and are working on validating the assay on a number of commercially available automated clinical diagnostic instruments, such as a Cobas c501 instrument from Roche, Olympus AU400 and AU480 instruments from Beckmann Coulter, Abbott Architect c4000 instrument, and Mindray BS-200E (RnD), BS-240, BS-380 and BS-400 instruments. In a first validation experiment, the assay was run on a Mindray BS-240.

For validation purposes, the inventors use true patient samples representing healthy volunteers as well as samples from patients diagnosed with an active virus infection, for example Covid-19, patients who have recovered from such infection. As a comparison, historical samples obtained and stored before the emergence of the virus in question, can be used, as well as artificial zero samples or blanks.

For control and calibration purposes, a number of samples ranging from high to low concentrations of antibodies against the chosen viral antigen were prepared, together with a sample of the same general composition but containing no antibody. The samples were tested to give expected results in one or more serological assays other than the homologous assay described herein. For example, an ELISA based assay was used to determine the concentrations of antibody in the samples.

### 3.1 Samples

Two types of biological materials were used in sample preparation, namely: human negative serum prior COVID-19 (from December 2019 or before) and human positive serum from prior infected individuals. The sample materials were purchased from CerbaXpert as a pool of positive samples assigned a high antibody response in a SARS-COV2-serological assay and a pool of negative samples from 2018, before the outbreak of Covid-19. The target concentration in the high pool was 10-20 mg/L. Low samples, and samples of concentrations between negative and high pool were prepared by mixing the pool of negative serum with a pool of high positive serum. Samples of higher concentrations were prepared by spiking serum with affinity purified anti human SARS-COV2-S1 (Innovagen AB, Sweden).

### 3.2 Precision

The precision study was performed using two natural samples of human positive serum, 2-4 mg/L (PR2) and 10-20 mg/L (PR-3). For Pr-3 positive pool was used as such and Pr-2 was prepared by diluting the positive pool 6 times with negative pool. Each sample was aliquoted, stored in freezer and used for all 5 runs over 3 days. The particles were prepared as described in Example 2.

Before each measurement, one aliquot of each sample was thawed on the bench. Visible precipitates were removed by centrifugation at 13 000 rpm (Heraeus, Biofuge A). Both precision samples (PR-2 and PR-3) were measured in 5 replicates over three days (N=25). Results for Pr-P2 and Pr-P3 are presented in Table 5 and Table 6. The total imprecision for Pr-2 and Pr-3 are below 10%.

TABLE 5
Results from 25 measurements performed in five
separate runs over three days on Pr-2 and Pr-3

| | Day 1 | Day 2 | Day 3 | Day 4 | Day 5 |
|---|---|---|---|---|---|
| Pr-2 | | | | | |
| Rep1 | 3.46 | 3.42 | 3.29 | 3.25 | 3.36 |
| Rep2 | 3.32 | 3.54 | 3.34 | 3.49 | 3.20 |
| Rep3 | 3.41 | 3.36 | 3.30 | 3.49 | 3.20 |
| Rep4 | 3.40 | 3.28 | 3.29 | 3.40 | 3.03 |
| Rep5 | 3.36 | 3.32 | 3.20 | 3.27 | 3.11 |
| Pr-3 | | | | | |
| Rep1 | 16.66 | 16.60 | 16.84 | 16.86 | 16.51 |
| Rep2 | 16.66 | 16.67 | 16.83 | 16.83 | 16.50 |
| Rep3 | 16.78 | 16.81 | 16.81 | 16.95 | 16.50 |
| Rep4 | 16.72 | 16.72 | 16.89 | 16.82 | 16.51 |
| Rep5 | 16.67 | 16.73 | 16.90 | 16.91 | 16.49 |

TABLE 6
Repeatability, between-run imprecision,
and total imprecision for Pr-2 and Pr-3

| | | Repeatability | | Between-run | | Total | |
|---|---|---|---|---|---|---|---|
| | Mean | SD | CV | SD | CV | SD | CV |
| | [mg/L] | [mg/L] | [%] | [mg/L] | [%] | [mg/L] | [%] |
| Pr-2 | 3.32 | 0.09 | 2.8 | 0.082 | 2.5 | 0.124 | 3.7 |
| Pr-3 | 16.73 | 0.05 | 0.3 | 0.148 | 0.9 | 0.157 | 0.9 |

### 3.4 on Board and Calibration Curve Stability

On board stability and calibration curve stability were performed using two natural anti-SARS-COV2-S1 serum samples having an anti-SARS-COV2-S1 concentration of 2-4 mg/l (Sample ID ST-1) and 10-20 mg/l (Sample ID ST-2) respectively. Each sample was aliquoted and stored in freezer. After thawing the samples prior to measurement, samples were centrifuged at 13000 rpm (Heraeus, Biofuge A) for 10 minutes and visible precipitates were removed. Each sample was measured in triplicates on each timepoint.

Results are shown in Table 7 and 8. Both on board and calibration curve stability pass the recovery and CV criteria after two weeks.

TABLE 7
Results of on-board stability study

| | Mean | | | |
|---|---|---|---|---|
| | concen- | % recovery | % recovery | Recovery |
| Sample ID | tration | from baseline | from baseline | and % CV |
| (Serum) | (mg/L) | week 1 | week 2 | pass/fail |
| ST-1 | 3.18 | 105 | 104 | Pass |
| ST-2 | 16.76 | 103 | 108 | Pass |

TABLE 8
Results of calibration curve stability study

| | Mean | | | |
|---|---|---|---|---|
| | concen- | % recovery | % recovery | % recovery |
| Sample ID | tration | from baseline | from baseline | and % CV |
| (Serum) | (mg/L) | week 1 | week 2 | pass/fail |
| ST-1 | 3.31 | 106 | 111 | Pass |
| ST-2 | 16.79 | 101 | 103 | Pass |

### 3.5 Measuring Range and Linearity

The linearity study was performed using two samples, one with human anti-SARS-COV2-S1 concentration 10-20 index units (Sample ID START-H) and one negative (Sample ID START-L). High pool (CerbaXpert) was used as Start-H and negative pool (CerbaXpert) as Start-L. Aliquots of Start-H and Start-L were stored frozen until use. After thawing at room temperature, the samples were centrifuged to remove precipitated material. A linearity dilution series was prepared by diluting sample START-H with sample START-L according to Table 9.

TABLE 9
Linearity dilution series

| Sample | START-H | START-L | START-H | START-L |
|---|---|---|---|---|
| ID | [%] | [%] | [μL] | [μL] |
| L1 | 100 | 0 | 600 | 0 |
| L2 | 90 | 20 | 540 | 60 |
| L3 | 80 | 30 | 480 | 120 |
| L4 | 70 | 50 | 420 | 180 |
| L5 | 60 | 70 | 360 | 240 |
| L6 | 50 | 90 | 300 | 300 |
| L7 | 40 | 93 | 240 | 360 |
| L8 | 30 | 95 | 180 | 420 |
| L9 | 20 | 97.5 | 120 | 480 |
| L10 | 10 | 98.5 | 60 | 540 |
| L11 | 5 | 100 | 30 | 570 |

The linearity was calculated as recovery for each sample L1-L11, measured in triplicate, and the results are presented in Table 10. The assay results in an acceptable recovery in the range from 1.3 to 11.4 index units. At lower concentrations, especially below the index cut-off value, CV increases due to modest calibration curve slope in the low concentration range. There was some overestimation of the sample antibody content, most prominent in the mid-range of the calibration curve. This could be due to the S-shaped calibration curve or the fact that the calibrator is based on rabbit IgG and the sample is human heterogenous serum containing IgA, IgG and IgM against the S1 spike protein, or a combination of them both.

TABLE 10
Results from linearity measurements

| Sample ID | START-H | START-L | Mean | SD | CV | Expected | Recovery |
|---|---|---|---|---|---|---|---|
| L1 | 600 | 0 | 11.17 | 0.12 | 1.03 | 11.4 | 98 |
| L2 | 540 | 60 | 10.47 | 0.06 | 0.55 | 10.3 | 102 |
| L3 | 480 | 120 | 9.77 | 0.06 | 0.59 | 9.2 | 106 |
| L4 | 420 | 180 | 8.93 | 0.06 | 0.65 | 8.0 | 112 |
| L5 | 360 | 240 | 8.00 | 0.10 | 1.25 | 6.9 | 116 |
| L6 | 300 | 300 | 6.90 | 0.10 | 1.45 | 5.8 | 119 |
| L7 | 240 | 360 | 5.60 | 0.10 | 1.79 | 4.7 | 119 |
| L8 | 180 | 420 | 4.23 | 0.06 | 1.36 | 3.5 | 121 |
| L9 | 120 | 480 | 2.87 | 0.21 | 7.26 | 2.4 | 119 |
| L10 | 60 | 540 | 1.47 | 0.06 | 3.94 | 1.3 | 113 |
| L11 | 30 | 570 | 0.77 | 0.12 | 1.06 | 0.7 | 110 |

### 3.7 Sensitivity-LoQ

LoQ was performed on two particle lots 20210316 (120 nm) and 20210316 (130 nm). For LoQ, a starting sample was assigned a concentration by running 6 replicates on the Mindray BS-240. From this start sample, two diluted samples for the LoQ study were prepared by diluting with negative serum. The study showed that LoQ of the assay is <1.5 mg/L on Mindray BS-240.

The results for LoQ measurements on Mindray BS-240 are presented in Table 11 and Table 12. Each LoQ sample was aliquoted in three vials and measured during 2 days with a total of 30 replicates on each LoQ sample.

TABLE 11
LoQ study on 120 nm particle lot

| | | | Bias from | |
|---|---|---|---|---|
| | | Theoretical | theoretical | |
| Sample | Mean | value | value | |
| material | (mg/L) | (mg/L) | (mg/L) | CV % |
| LoQ - 1 | 2.19 | 2.59 | 0.40 | 12.5 |
| LoQ - 2 | 1.48 | 1.73 | 0.25 | 15.3 |

TABLE 12
LoQ study on 130 nm particle lot

| | | | Bias from | |
|---|---|---|---|---|
| | | Theoretical | theoretical | |
| Sample | Mean | value | value | |
| material | (mg/L) | (mg/L) | (mg/L) | CV % |
| LoQ - 1 | 1.80 | 2.08 | 0.28 | 6.4 |
| LoQ - 2 | 1.00 | 1.38 | 0.38 | 13.6 |

### 3.8 Limit of Blank

Since the qualitative question “are there any antibodies?” is of interest, limit of blank was also investigated. 40 negative samples (serum collected before 2019) were used for LoB determination. Tukey outlier test (K=3) excluded one negative sample from the LoB calculation but is included in specificity determination. LoB was calculated with both a 95% and 99% confidence interval. Results are shown in Table 13.

TABLE 13
LoB and LoD based on 39 negative samples (serum collected prior to 2019)

| | Mean | | | LoB₉₅ | | LoB₉₉ |
|---|---|---|---|---|---|---|
| Lot | response* | STDEV | LoB₉₅** | μg/mL*** | LoB₉₉**** | μg/mL*** |
| 20210316 | 22.82 | 15.48 | 53.16 | 1.26 | 62.69 | 1.47 |
| (120 nm) | | | | | | |
| 20210316 | −1.69 | 14.53 | 26.80 | 0.58 | 35.75 | 0.74 |
| (130 nm) | | | | | | |

*AU response on Mindray BS-240 for negative samples
**Mean + 1.96*STDEV (95% CI)
***AU response in μg/mL based on 3 different calibration curves
****Mean + 2.576*STDEV (99% CI)

### 3.9 Clinical Performance—Sensitivity Specificity

40 positive and 40 negative samples were purchased from CerbaXpert. The positive samples were confirmed positive for S1-antibodies in SARS-COV-2 IgG (Architect) and SARS-COV-2-IgM (Architect). Both assays are chemiluminescent microparticle immunoassays (CMIA) using the Spike protein as antigen.

As cut off value for the Gentian turbidimetric assay we used LoB95 (Mean blank+1.96×Stdev blank, one sided 95% confidence interval) or LoB99 (Mean blank+2.576×Stdev blank, one sided 99% confidence interval). LoB95 was determined to 1.26 μg/ml and LoB99 to 1.47 μg/ml for the particle LOT 20210316-120 nm. For 20210316-130 nm LoB was lower, but the results in terms of positive/negative was the same for all samples.

Moreover, the results (positive/negative) from the measurements on the in total 80 samples were the same when using LoB95 and LOB99 as cut-off value. The results are presented in Table 14 and all calculations performed according to EP12. The estimated sensitivity was 97.6% (84.2-103.1) and the estimated specificity 97.5% (85.7-101.0) see Table 15.

TABLE 14
Results from measurements on 40 positive and 40 negative samples

| | Positive | Negative | Total |
|---|---|---|---|
| Positive | 41 | 1 | 42 |
| Negative | 1 | 39 | 40 |
| Total | 42 | 40 | 82 |

TABLE 15
Calculations performed according to EP12
Results from the study

| | |
|---|---|
| Estimated sensitivity | 97.6% |
| Estimated specificity | 97.5% |
| Disease prevalence | 51.2% |
| Study predictive value of a positive result | 97.6% |
| Study predictive value of a negative result | 97.5% |

Calculations for 95% score confidence limits (EP12 A2E)

| | |
|---|---|
| Q1, se | 85.8 |
| Q2, se | 5.5 |
| Q3, se | 91.7 |
| Lower limit sensitivity | 87.7% |
| upper limit sensitivity | 99.6% |
| Q1, sp | 81.8 |
| Q2, sp | 5.5 |
| Q3, sp | 87.7 |
| lower limit specificity | 87.1% |
| upper limit specificity | 99.6% |

### 3.10 Security Zone

Turbidimetric assay are usually affected by an excess effect of the analyte to be measured. This means that the turbidity in the sample, at some point, decreases with increasing concentration of the analyte. Therefore, the assay might return a false low value at some concentration level, but as long as the returned concentration is above the programmed linear range or highest standard in calibration curve, the instrument will detect that this is sample outside the calibration range and rerun the sample in diluted mode and return the correct concentration in a second run. The security zone can be manipulated in many ways in the application settings (reading time, volumes, wavelength).

During testing on Mindray BS-240, it was observed that the current assay can manage a hook sample at a concentration of at least 100 mg/L. In this experiment, 40 positive samples and 2 samples from vaccinated patients (vaccinated with Pfizers vaccine) were tested (see section 5.6 and 5.3 Selection of antibody). Out of these 42 samples 5 were flagged as being higher than the highest calibrator on both the 120 nm and 130 nm lot (both vaccinated samples and 3 other positive samples). These 5 samples were tested with a 1:10 dilution and no sample were higher than the hook sample, see FIG. 7. The figure clearly shows that the assay can manage high samples without a hook effect, and it is assumed that it will be able to handle samples up to at least 100 mg/L

### Example 4. Optimization of Particle Size and Wavelength

The protocol disclosed in Examples 1 and 2 is first repeated using particles having a diameter of 120 nm, and the absorption tested at various wavelengths (such as 340 nm, 380 nm, 560 nm, up to 670 nm). The above protocol is then repeated using particles having a diameter of 150 nm, and the absorption tested at various wavelengths (such as 340 nm, 380 nm, 560 nm, and up to 670 nm). Further examples can be conducted using particles of a smaller or larger diameter, using the same protocol.

Additional experimentation may be required to determined optimal concentrations of the reagents, duration of different steps and physical and chemical parameters.

In summary, the inventors have made available robust and reliable assay which is suitable for mass testing and makes it practically and economically feasible to monitor the spread of an infection in a population and the development of immunity, and also to investigate effects of a vaccination regime. The invention makes it possible to measure antibodies in a sample using open spectrophotometric instrument platforms that are already in use all clinical chemistry laboratories. Examples include, but are not limited to, the Cobas instrument range from Roche, the Olympus AU instruments from Beckman Coulter, Mindray spectrophotometric instruments for measurements of turbidimetry and numerous other instruments. As these instruments are widely in use, an assay according to the present invention can be put to use immediately, without the need for acquiring new instruments.

Without further elaboration, it is believed that a person skilled in the art can, using the present description, including the examples, utilize the present invention to its fullest extent. Also, although the invention has been described herein with regard to its preferred embodiments, which constitute the best mode presently known to the inventors, it should be understood that various changes and modifications as would be obvious to one having the ordinary skill in this art may be made without departing from the scope of the invention which is set forth in the claims appended hereto.

Thus, while various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for detection of an antibody against a virus in a sample suspected of comprising antibodies against said virus, comprising:
contacting the sample with a nanoparticle having capture molecules and co-molecules immobilized thereon, wherein a capture molecule comprises a peptide that is specifically recognized by the antibody and wherein a co-molecule is not specifically recognized by the antibody and has a molecular weight smaller than the molecular weight of the capture molecule, wherein the co-molecules when immobilized on said nanoparticle separate the capture molecules resulting in an average distance between two adjacent capture molecules being greater than a distance between antigen binding sites of the anti-virus antibody to be detected
forming a complex of the antibody with the capture molecule immobilized on the nanoparticle in the presence of the antibodies in the sample, and
determining a signal indicative for the presence of the complex, wherein the signal is measured by turbidimetry or nephelometry.

2. The method according to claim 1, wherein said capture molecule is an epitope isolated from a spike, envelope, membrane or nucleocapsid protein of said virus.

3. The method according to claim 1, wherein said virus is a SARS-CoV-2 virus including variants thereof.

4. The method according to claim 1, wherein the co-molecule has a primary amine moiety.

5. The method according to claim 1, wherein the co-molecule is chosen from the group comprising hydroxylamine, tris(hydroxymethyl)aminomethane, amino acids, low molecular weight peptides and ethanolamine.

6. The method according to claim 1, wherein the co-molecule is ethanolamine.

7. The method according to claim 1, wherein the co-molecule has a molecular weight in the range of 50-1500 Da.

8. The method according to claim 1, wherein the co-molecule is a peptide having a molecular weight in the range of 300-1500 Da.

9. The method according to claim 1, wherein the sample is a sample chosen from mammalian body fluids such as mucus (nasal or laryngeal), sputum, saliva, tears, feces (including fecal extracts), urine, whole blood or a blood derived sample like blood plasma or blood serum.

10. A kit for detection by turbidimetry or nephelometry of antibodies against a virus in a sample, comprising at least one specific capture molecule for an anti-virus antibody and a co-molecule immobilized to a nanoparticle, wherein two or more such capture molecules are immobilized on each particle, characterized in that
said capture molecule comprises an epitope isolated from a spike, envelope, membrane or nucleocapsid protein of said virus,
said co-molecule is not specifically recognized by the antibody and has a molecular weight smaller than that of said capture molecule,
said co-molecules when immobilized on said monodisperse particles separate said capture molecules resulting in that an average distance between two adjacent capture molecules is greater than a distance between antigen binding sites of the anti-virus antibody to be detected.

11. The kit according to claim 10, wherein said virus is a SARS-CoV-2 virus including variants thereof.

12. The kit according to claim 10, wherein said virus is a SARS-CoV-2 virus including variants thereof and the capture molecule comprises an epitope isolated from a spike protein of said virus,

13. The kit according to claim 10, wherein the co-molecule has a primary amine moiety.

14. The kit according to claim 10, wherein the co-molecule is chosen from the group comprising hydroxylamine, tris(hydroxymethyl)aminomethane, amino acids, low molecular weight peptides and ethanolamine.

15. The kit according to claim 10, wherein the co-molecule is ethanolamine.

16. The kit according to claim 10, wherein the co-molecule has a molecular weight in the range of 50-1500 Da.

17. The kit according to claim 10, wherein the co-molecule is a peptide having a molecular weight in the range of 300-1500 Da.

18. The kit according to claim 10, wherein said nanoparticle has a diameter in the range of 10-300 nm.

19. A nanoparticle as part of a kit according to claim 10, comprising a capture molecule chosen from an epitope isolated from a spike, envelope, membrane or nucleocapsid protein of a virus, and a co-molecule immobilized to said particle, wherein said co-molecule is not specifically recognized by the antibody and has a molecular weight smaller than that of said capture molecule, said co-molecules when immobilized on said monodisperse particles in an amount greater than the amount of capture molecules separating said capture molecules resulting in that an average distance between two adjacent capture molecules is greater than a distance between antigen binding sites of the anti-virus antibody to be detected.

20. The nanoparticle according to claim 19, wherein said virus is a SARS-CoV-2 virus including variants thereof.

21. The nanoparticle according to claim 19, wherein said virus is a SARS-CoV-2 virus including variants thereof, and the capture molecule is an epitope isolated from a spike protein of said virus.

22. The nanoparticle according to claim 19, wherein said co-molecule molecule has a primary amine moiety.

23. The nanoparticle according to claim 19, wherein said co-molecule is chosen from the group comprising hydroxylamine, tris(hydroxymethyl)aminomethane, amino acids, low molecular weight peptides and ethanolamine.

24. The nanoparticle according to claim 19, wherein the co-molecule is ethanolamine.

25. The nanoparticle according to claim 19, wherein the co-molecule has a molecular weight in the range of 50-1500 Da.

26. The nanoparticle according to claim 19, wherein the co-molecule is a peptide having a molecular weight in the range of 300-1500 Da.

27. The nanoparticle according to 6 claim 19, wherein said nanoparticle has a diameter in the range of 10-300 nm.

28. A reagent solution comprising a nanoparticle according to claim 19.
